(19) United States
(12) Patent Application Publication
(10) Pub. No.: US 2024/0024291 A1
(43) Pub. Date: Jan. 25, 2024

(54) Uses of Dihydroorotate Dehydrogenase Inhibitors

(71) Applicant: University of Maryland, Baltimore, Baltimore, MD (US)

(72) Inventor: Eli E. Bar, Sykesville, MD (US)

(73) Assignee: University of Maryland, Baltimore, Baltimore, MD (US)

(21) Appl. No.: 18/354,910

(22) Filed: Jul. 19, 2023

Related U.S. Application Data
(60) Provisional application No. 63/390,473, filed on Jul. 19, 2022.

(51) Int. Cl. A61K 31/4196; A61K 45/06; A61P 35/00

(52) U.S. Cl. CPC A61K 31/4196; A61K 45/06; A61P 35/00

(57) ABSTRACT

Provided herein are methods for treating brain cancers, such as malignant gliomas, particularly glioblastomas. Inhibitors of dihydroorotate dehydrogenase singly or in combination with at least other one small molecule drug acting synergistically with the dihydroorotate dehydrogenase inhibitor are administered to a subject with a brain cancer or brought into contact with brain cancer tumor cells. Administration of a drug regimen of the inhibitor and at least one of the small molecule drugs increases sensitivity of a malignant glioma thereto.

### CROSS-REFERENCE TO RELATED APPLICATIONS

This non-provisional application claims the benefit of priority under 35 U.S.C. § 119(e) of provisional application U.S. Ser. No. 63/390,473, filed Jul. 19, 2022, the contents of which is hereby incorporated by reference in its entirety.

### Federal Funding Legend

This invention was made with government support under Grant Numbers CA187780 and NS106553 awarded by the National Institutes of Health. The government has certain rights in the invention.

### BACKGROUND OF THE INVENTION

### Field of the Invention

The present invention relates to the fields of neurology and oncogenic neuropatholology. More particularly, the present invention relates to methods for reducing the growth of gliomas, such as glioblastomas.

### Description of the Related Art

Glioblastoma (GBM) is the most common and lethal primary brain tumor in adults (1). After diagnosis, median survival is less than 15 months with maximal safe surgical resection, radiation, and temozolomide chemotherapy (2).

The challenges inherent in developing more effective treatments for glioblastoma have become increasingly apparent, including extensive brain invasion, resistance to standard treatments, genetic complexity, shielding by the blood-brain-barrier, and subpopulations of tumor cells with phenotypic similarities to embryonic stem cells, often referred to as glioma stem cells (GSC). Indeed, mounting evidence points to the relative chemoradiation resistance of glioma stem cells in glioblastoma and similar cells in tumors outside of the CNS (3-20). One critical factor affecting chemotherapy resistance is the hypoxic tumor microenvironment found in essentially all advanced glioblastoma tumors (21). Indeed, palisading necrosis and hypoxia-induced vascular proliferation are central to the World Health Organization (WHO) pathological classification of these tumors (22).

Metabolic reprogramming and plasticity enables cancer cells to adapt to harsh microenvironmental conditions during cancer progression and anti-tumor treatments. Metabolic reprogramming is intimately linked to glioblastoma therapy resistance (21), and thus, identifying cancer-specific metabolic dependencies, which limit metabolic adjustments by the tumor, represents an attractive therapeutic approach as it may improve the ability to eliminate glioma stem cells and their more differentiated progenies. Indeed, hypoxia regulates glioma stem cells in glioblastoma (23-28). It was recently demonstrated that reduced de novo pyrimidine synthesis, one of the most notable metabolic changes caused by the depletion of monocarboxylate transporter-4 (MCT4), significantly blocked the growth of glioblastoma neurosphere models in vitro and in vivo as orthotopic xenografts in immune-deficient mice (29). MCT4 depletion reprograms cancer metabolism from glycolysis to anaplerotic glutaminolysis. Such metabolic adjustments were mediated by reduced CAD (carbamoyl-phosphate synthetase 2, aspartate transcarbamylase, and dihydroorotase) and DHODH (dihydroorotate dehydrogenase) expression, the latter representing the rate-limiting step in the de novo pyrimidine synthesis pathway. De novo biosynthesis of pyrimidine nucleotides could represent a targetable metabolic vulnerability in some glioblastoma tumors.

The prior art is deficient in effective strategies to reduce the growth of glioblastoma cells. The present invention fulfills this need and desire in the art.

### SUMMARY OF THE INVENTION

The present invention is directed to a method for treating a brain cancer in a subject in need thereof. In this method, a pharmacologically effective amount of an inhibitor of dihydroorotate dehydrogenase is administered at least once to the subject. The present invention is directed to a related method further comprising administering to the subject a pharmacologically effective amount of at least one small molecule drug or pharmaceutically acceptable compositions thereof synergistic with the inhibitor. The present invention is directed to another related method further comprising administering at least once to the subject a chemoradiation therapy.

The present invention is further directed to a method for decreasing proliferation of malignant glioma tumor cells. In this method, the glioma tumor cells are contacted with at least one small molecule drug that inhibits an activity of dihydroorotate dehydrogenase or an activity associated therewith. The present invention is directed to a related method further comprising administering at least once to the subject a chemoradiation therapy.

The present invention is directed further to a method for increasing sensitivity of a malignant glioma to a drug regimen for a subject in need thereof. In this method, a regimen of compound 1 with a chemical structure of:

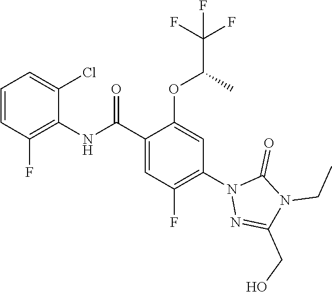 [SMILES: CCn1c(CO)nn(-c2cc(O[C@@H](C)C(F)(F)F)c(C(=O)Nc3c(F)cccc3Cl)cc2F)c1=O]

and at least one of a small molecule drug that synergizes therewith or pharmaceutically acceptable compositions thereof are administered at least once to a subject in need thereof.

Other and further aspects, features, benefits, and advantages of the present invention will be apparent from the following description of the presently preferred embodiments of the invention given for the purpose of disclosure.

### BRIEF DESCRIPTION OF THE FIGURES

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1G shows that compound 1 inhibits glioblastoma neurosphere growth in vitro with normalized response to compound 1 in the glioblastoma neurosphere lines 913, 08-387, 3691, 1079, Mayo6, Mayo15, and Mayo39, respectively.

FIGS. 2A-2D show growth inhibition by compound 1 can be rescued by uridine supplementation. CellTiter-Glo® assay for 913 (FIGS. 2A-2B) and 08-387 (FIGS. 2C-2D) glioblastoma neurospheres cultured in normoxia (21% oxygen, FIGS. 2A, 2C) and hypoxia (1% oxygen, FIGS. 2B, 2D) and treated with increasing concentrations of compound 1 alone (left half of each panel) or in combination with 100 mM uridine (right half of each panel). Statistics: One way ANOVA ** p<0.01, *** p<0.001, **** p<0.0001.

FIGS. 3A-3H shows that compound 1 inhibition of de novo pyrimidine synthesis promotes DNA damage in normoxia and hypoxia. Immunofluorescent staining for gH2AX foci in four glioblastoma neurosphere lines, 913 (FIG. 3A), 3691 (FIG. 3C), 08-387 (FIG. 3E), and 1079 (FIG. 3G) cultured in normoxia (21% oxygen) or hypoxia (1% oxygen) and treated with model-specific IC50 concentration of compound 1 for 48 hours. Images displayed show a composite of g-H2AX (green) and DAPI nuclear staining. Spot counting was performed with Gen5 software to identify and enumerate g-H2AX foci in all imagery (40× magnification). For convenience, an inset showing a single representative nucleus is shown on the bottom right of each of FIGS. 3A, 3C, 3E, and 3G. FIGS. 3B, 3D, 3F, and 3H are bar graphs summarizing the analysis for respective FIGS. 3A, 3C, 3E, and 3G. This set of experiments was performed at least twice on each of the glioblastoma models shown. Statistics: One-way ANOVA with Bonferroni correction. **p<0.01, ***p<0.001, ****p<0.0001.

FIGS. 4A-4M show that compound 1 induces early S phase cell cycle arrest. FIG. 4A: An illustration showing the structure of the PIP-FUCCI degron (Addgene Plasmid #118616), including the fusion between mVenus-Cdt and Geminin-mCherry (top), a key for the cell cycle distribution (middle), and an illustration of the PIP-FUCCI degron fusion proteins as they relate to cell cycle progression (bottom). 913 PIP-FUCCI reporter cells were treated with DMSO, 10 nM, and 100 nM compound 1 alone (FIGS. 4B-4D). Aphidicolin (3 mM) was used as a control for S-phase arrest (FIG. 4E). The effects of compound 1 were rescued with 100 mM uridine (FIGS. 4F-4G). Accurate delineation of cell cycle phase transition was monitored in living cells with direct fluorescence imaging and is indicated at the top left corner of panels (FIGS. 4B-4G). Representative images of each treatment are shown in panels (FIGS. 4H-4M). Scale Bar - 10 mm.

FIGS. 5A-5H show that compound 1 inhibits colony growth in NSMC. 913 and 08-387 neurosphere lines grown under normoxia (FIGS. 5A, 5C) or hypoxia (FIGS. 5B, in NSMC medium and treated with 0.1 nM compound 1 (grey bars), 1 nM compound 1 (pink bars), or 10 nM compound 1 (red bars) showed decreased sphere count and size compared with DMSO (black bars) treated cells (*** P<0.001, **** P<0.0001, one-way ANOVA with Dunnett's multiple comparisons test). Representative images of treated neurospheres are shown (FIGS. 5E-5H). Scale Bar - 250 mm.

FIG. 6 shows that compound 1 induces Poly(ADP-ribose) polymerase (PARP) cleavage. Western blots showing BAY2402234 induces programmed cell death as indicated by increased cleavage of PARP. The values shown depict the relative levels of cleaved PARP.

FIGS. 7A-7H show that EGFR signaling promotes compound 1 resistance in glioblastomas and that compound 1 promotes sensitivity to gemcitabine in glioblastomas. FIG. 7A: ERK and AKT are constitutively phosphorylated in compound 1-resistant but not responsive GBMs. FIG. 7B: EGFRvIll expression promotes compound 1 resistance in 913 and 08-387 GSCs. FIGS. 7C-7D: Nonlinear fit curves of 08-387 GFP and 913 GFP, respectively, show EGFRvii drives resistance to compound 1. FIGS. 7E-7F: Nonlinear fit curves of 08-387 GFP and 913 GFP, respectively, show sensitivity to gemcitabine. FIGS. 7G-7H: Nonlinear fit curves of EGFRviii expressing 08-387 GFP and 913 GFP, respectively, show sensitization to gemcitabine. Statistics: Student t-test **** p<0.0001

FIGS. 8A-8F shows that compound 1 slows the growth of orthotopic glioblastoma xenografts and prolongs survival. Animals with 913 (FIGS. 8A, 8C) or 08-387 (FIGS. 8B, 8D) xenografts were separated into treatment and control groups. When the Radiance of luciferase-expressing tumors surpassed 1x10 6, daily oral treatment with compound 1 or vehicle was initiated (designated t=0). Tumors from each group were imaged once per week for up to five weeks from the initiation of treatment, and all animals were sacrificed when they became symptomatic. Daily average body weight is shown in panels (FIG. 8A, 913) and (FIG. 8B, 08-387). Comparison of tumor Radiance on week 1 through 3 for 913 (FIG. 8C) and 08-387 (FIG. 8D) (** p<0.01, mixed-effects model analysis with . 8 idak's multiple comparisons test). compound 1-treated mice have significantly prolonged survival (median survival: 41 vs. 32 days in 913 (FIG. 8E) and 19 vs. 12 days in 08-387 (FIG. 8F) from initiation of treatment, log-rank test: ** p<0.01, **** p<0.0001).

FIGS. 9A-9B shows that Mayo 6 GBMs (FIG. 9A) and Mayo 39 GBMs (FIG. 9B) are moderately sensitized to DHODHi by Sorafenib. Analysis is performed using SynergyFinder.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

The term “pharmacologically effective amount” as used herein refers to generally an amount effective to accomplish the intended purpose, for example, but not limited to, treating a brain cancer, reducing resistance to or increasing sensitivity of a drug. However, the amount can be less than that amount when a plurality of drugs or therapeutically active agents are to be administered, i.e., the total effective amount can be administered in cumulative dosage units. The amount of drug, small molecule drug or therapeutically active agent may also be more than the pharmacologically effective amount when the drug or therapeutically active agent provides sustained release of the pharmacologically active agent comprising the same. The total amount of a pharmacologically active agent to be used can be determined by methods known to those skilled in the art. However, because the drugs or therapeutically active agent may deliver the pharmacologically active agent more efficiently than prior drugs, active agents or pharmaceutical compositions, less amounts of active agent than those used in prior dosage unit forms or delivery systems can be administered to a subject while still achieving the same levels in the brain and/or therapeutic effects.

As used herein, the term “pharmaceutically acceptable” refers to molecular entities and compositions that do not produce an adverse, allergic or other untoward reaction when administered to a subject, such as, for example, a human or non-human mammal, as appropriate. The preparation of a pharmaceutical composition that contains an inhibitor, an antagonist or compound is known to those of skill in the art in light of the present disclosure, as exemplified by Remington's Pharmaceutical Sciences, 18th Ed. Mack Printing Company, 1990.

As used herein, the term “contacting” refers to any suitable method of bringing a drug, small molecule drug, therapeutically active agent, compound or a pharmaceutical composition into contact with a cell in vivo, in vitro or ex vivo. For in vivo applications, any known method of administration suitable for a subject with a brain cancer, such as a glioma, particularly a glioblastoma, as described herein.

As used herein, the term “subject” refers to any recipient, for example a human or non-human mammal, of the drugs, small molecule drugs, therapeutically active agents, compounds and/or pharmaceutical compositions as described herein singly, in combination or with another treatment for a brain cancer, such as radiation.

In one embodiment of the present invention, there is provided a method for treating a brain cancer in a subject in need thereof, comprising administering at least once to the subject a pharmacologically effective amount of an inhibitor of dihydroorotate dehydrogenase.

Further to this embodiment, the method comprises administering to the subject a pharmacologically effective amount of at least one small molecule drug or pharmaceutically acceptable compositions thereof synergistic with the inhibitor. In this further embodiment, representative small molecule drugs include but are not limited to gemcitabine, sorafenib, vandetanib, vemurafenib, regorafenib, or osimertinib. Also in both embodiments, the one small molecule drug is co-administered with the inhibitor or is administered sequentially therewith. In another further embodiment, the method comprises administering at least once to the subject a chemoradiation therapy.

In both embodiments, the inhibitor of dihydroorotate dehydrogenase may be compound 1 with a chemical structure of:

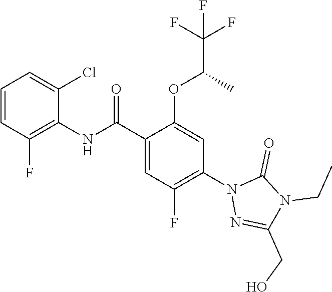 [SMILES: CCn1c(CO)nn(-c2cc(O[C@@H](C)C(F)(F)F)c(C(=O)Nc3c(F)cccc3Cl)cc2F)c1=O]

or a pharmaceutically acceptable composition thereof. Also in both embodiments, the brain cancer may be a malignant glioma. Particularly the malignant glioma may be a glioblastoma. In aspects thereof, the glioblastoma may overexpress EGFR or mutant EGFRviii.

In another embodiment of the present invention, there is provided a method for decreasing proliferation of malignant glioma tumor cells, comprising contacting the glioma tumor cells with at least one small molecule drug that inhibits an activity of dihydroorotate dehydrogenase or an activity associated therewith. In a further embodiment, the method comprises administering a chemoradiation therapy at least once to the subject.

In one aspect of this embodiment, the contacting step is in vivo, and the method comprises administering at least once to a subject in need thereof a pharmacologically effective amount of the small molecule drug. In this aspect, the small molecule drug is compound 1 with the chemical structure as described supra or a pharmaceutically acceptable composition thereof. Also in this aspect, the small molecule drug may comprise a combination of compound 1 and at least one other small molecule drug synergistic therewith or a pharmaceutically acceptable composition of the other small molecule drug. Representative examples of the other small molecule drug is at least one of gemcitabine, sorafenib, vandetanib, vemurafenib, regorafenib, or osimertinib.

In another aspect of this embodiment, the contacting step is in vitro, and the method comprises bringing the malignant glioma tumor cells into contact with compound 1 or a combination of compound 1 and at least one of gemcitabine, sorafenib, vandetanib, vemurafenib, regorafenib, or osimertinib.

In this embodiment and both aspects thereof, the malignant glioma tumor cells may comprise a glioblastoma. In an aspect thereof, the glioblastoma may overexpress EGFR or mutant EGFRviii.

In yet another embodiment of the present invention, there is provided a method for inhibiting increasing sensitivity of a malignant glioma to a drug regimen for a subject in need thereof, comprising administering to the subject at least once a regimen of compound 1 with a chemical structure of:

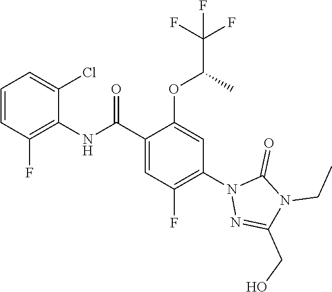 [SMILES: CCn1c(CO)nn(-c2cc(O[C@@H](C)C(F)(F)F)c(C(=O)Nc3c(F)cccc3Cl)cc2F)c1=O]

and at least one of a small molecule drug that synergizes therewith or pharmaceutically acceptable compositions thereof. In this embodiment, the at least one small molecule drug is gemcitabine, sorafenib, vandetanib, vemurafenib, regorafenib, or osimertinib. Also in this embodiment, the drug regimen may comprise co-administering or sequentially administering compound 1 and the at least one small molecule drug or the pharmaceutically acceptable compositions thereof. In addition, the malignant glioma may be a glioblastoma. In an aspect thereof, the glioblastoma may overexpress EGFR or mutant EGFRviii.

Provided herein are methods for treating a brain cancer, such as a malignant glioma, particularly a glioblastoma and methods for decreasing the proliferation thereof in vivo and in vitro. The glioblastoma may overexpress EGFR or mutant EGFRviii and, as such, a method for increasing sensitivity of malignant glioma to one or more inhibitors and/or other small molecule drugs.

The inhibitors, such as in a non-limiting example, an inhibitor of dihydroorotate dehydrogenase or an activity thereof or an activity associated therewith is compound 1 (BAY2402234) with a structure as shown herein. Other inhibitors or small drug molecules that exhibit synergistic effects with compound 1 include, but are not limited to, gemcitabine, sorafenib, vandetanib, vemurafenib, regorafenib, or osimertinib.

In the methods provided herein, one of ordinary skill in the art is well able to determine a drug regimen for treatment of a subject with a brain cancer, for example, a malignant glioma, particularly a glioblastoma, such as, but not limited to, one that overexpresses EGFR or mutant EGFRviii. Determination of the dosage of the DHODH inhibitor and of the one or more other small molecule drugs, the route of administration and administration schedule depends at least on the age and sex of the subject, the general health thereof, whether or not the subject has had other chemotherapeutic interventions, for example, chemoradiation of the tumor, and the prognosis derived therefrom. Methods of administration are well-known in the art and one of ordinary skill in the art is well able to select and utilize the same.

The following examples are given for the purpose of illustrating various embodiments of the invention and are not meant to limit the present invention in any fashion.

### Example 1

Methods and Materials Chemicals and Reagents

Compound 1 (BAY2402234; (N-(2-chloro-6-fluorophenyl)-4-[4-ethyl-4,5-dihydro-3-(hydroxymethyl)-5-oxo-1H-1,2,4-triazol-1-yl]-5-fluoro-2-[(1S)-2,2,2-trifluoro-1-methylethoxy]-benzamide):

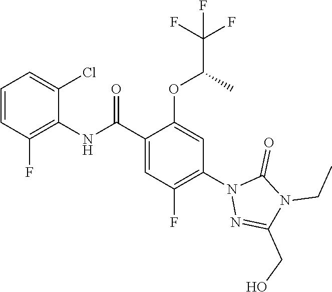 [SMILES: CCn1c(CO)nn(-c2cc(O[C@@H](C)C(F)(F)F)c(C(=O)Nc3c(F)cccc3Cl)cc2F)c1=O]

was obtained from BAYER (Leverkusen, Germany). For animal studies, compound 1 was prepared by dissolving the required amount of active compound in 90/10 Kollisolv® PEG400/EtOH solution at 50° C. with continuous stirring.

pLenti-PGK-Neo-PIP-FUCCI was obtained from Jean Cook (Addgene plasmid #118616; http://n2t.net/addgene:118616; RRID:Addgene_118616). Cloning of the pLV[Expressior]-mCherry:T2A:Hygro-EF1A>Luc2 was outsourced to VectorBuilder. The plasmid is available on Addgene, plasmid #174665; RRID:Addgene_174665. Donkey Anti-Mouse IgG (H+L) Antibody, Alexa Fluor 488 Conjugated (Molecular Probes Cat #A-21202, RRID:AB_141607) was used at 1:250 dilution in immunofluorescence studies.

### Glioblastoma Neurosphere Lines

The glioblastoma neurosphere line 913 was a gift from Dr. Angelo Vescovi (Hospital San Raffaele, Milano, Italy). The glioblastoma neurosphere lines 08-387 and 3691 were provided by Dr. Jeremy N. Rich (UPMC Hillman Cancer Center). Dr. Ichiro Nakano (the University of Alabama at Birmingham, AL) provided the 1079 line. Finally, the Mayo6, Mayo15, and Mayo39 models were acquired from Dr. Jann N. Sarkaria, who manages the Mayo Clinic Brain Tumor Patient-Derived Xenograft (PDX) National Resource. Each line was verified by short tandem repeat analysis (STR) and regularly tested for mycoplasma. The lines were maintained in defined neural stem cell medium (see formulation below).

### Culturing Glioblastoma Neurospheres in Hypoxia

A Biospherix X3, hypoxic incubation system, was maintained at 37° C., 1% 02, 5% CO2, and 94% N2 (Biospherix, Ltd—Parish, NY) to conduct in vitro hypoxic experiments. Cells were plated, then incubated in normoxia overnight before transferring the culture dishes to hypoxia for the duration indicated in the main text.

### In Vitro Proliferation Assays

Glioblastoma neurosphere lines were plated in 96-well plates at 25,000 cells per well in 100 pL of media and treated with compound 1 in the presence or absence of 100 μM uridine. Cell Titer-Glo® Luminescent Cell Viability Assay (Promega, Madison, WI, USA) was performed 72 hours after treatment. Luminescence was determined using a Cytation 5 reader and Gen5 software package (BioTek, Winooski, VT, USA).

### Resistance and Sensitization Assays to Compound 1

Ten thousand 08-387 or 913 glioblastoma cells per well were seeded in a 96 well plate. Post 24 hr plating, 08-387 and 913 cells were treated with a varying dosage of compound 1 or with a varying dosage of gemcitabine alone or with 100 nM of compound 1. Post 72 hr of treatment, cell viability was calculated on Alamar blue absorbance reading.

Growth Assays in Neurosphere Media Supplemented with Methylcellulose

Glioblastoma neurosphere cells were seeded in 1 mL of neuro-stem-cell medium supplemented with 0.375% methylcellulose (NSMC) containing 0, 0.1 nM, or 1 nM compound 1 into 12-multiwell culture plates (10,000 cells/well), then cultured in normoxia (21% 02) or hypoxia (1% 02) for seven to ten days. Cells were fed with 0.2 mL NCMS medium containing compound 1 at the indicated concentration every other day. Colony number and size were determined with the Gen5 Image Prime software package by analyzing brightfield images acquired with a BioTek Cytation 5 instrument equipped with 4X and 20X objectives.

### Defined Neuro-Stem-Cell Medium (NSM) Formulation

DMEM (Dulbecco's Modified Eagle's Medium)/Hams F-12 50/50 Mix (Corning #10-090-CV), 20 ng/mL Animal-Free Recombinant Human EGF (Peprotech #AF-100-and 10 ng/mL Animal-Free Recombinant Human FGF-basic (154 a.a.) (Peprotech AF10018B), 1×Corning® ITS (Insulin-Transferrin-Selenium), 0.2% Bovine Serum Albumin (BSA), Fraction V (GeminiBio #700-100P), 0.0002% Heparin (w/v, Sigma H-3149), 4.8 mM Putrescine dihydrochloride (Sigma, P5780), and 0.016 mM Progesterone (Sigma P8783). Sterilize by filtration through a Nalgene™ Rapid-Flow™ Bottle Top Filter (Thermo Scientific™ Catalog number 596-3320).

### Western Blot Analyses

Western Blots were performed using 25 pg of protein per sample loaded onto NuPAGE® Novex® 4-12% Bis-Tris protein gels and transferred onto nitrocellulose membranes that were then blocked with 5% BSA in PBS and 0.1% Tween (PBST). Primary antibody incubation was performed in 5% BSA in PBST at 4° C. overnight, and HRP-labeled secondary antibody incubation (1:5000) was performed for one hour at room temperature in 5% BSA in PBST.

Antibodies used for western blotting were acquired from the following sources: Cleaved PARP (Asp214) (D64E10) XP® Rabbit mAb, Cell Signaling Technology Cat#5625, RRID:AB_10699459, 1:1000 for Western blots. Phospho-H2AX, Millipore Cat#RRID:AB_2755003, 1:1000 for Western blots, and 1:250 for immunofluorescence imaging. p-Tubulin, Millipore Cat #MAB5564, RRID:AB 11212768, 1:10,000 for Western blots. p44/42 MAPK (Erk1/2) (137F5) Rabbit mAb (Cell Signaling Technology Cat #4695, RRID:AB_390779) 1:1000 for Western blots; Phospho-p44/42 MAPK (Erk1/2) (Thr202/Tyr204) (Cell Signaling Technology Cat #9101, RRID:AB_331646) 1:1000 for Western blots; Akt Antibody (Cell Signaling Technology Cat #9272, RRID:AB_329827) 1:1000 for Western blots; Phospho-Akt (Ser473) Antibody (Cell Signaling Technology Cat #9271, RRID:AB 329825) 1:1000 for Western blots.

### Lentivirus Plasmids

The pLV[Expressior]-mCherry:T2A:Hygro-EF1A>Luc2 (Addgene Plasmid #174665) and pLV[Exp]-Bsd-EFS>EGFRvIll:T2A:TurboGFP (Addgene Plasmid #189841) lentivirus plasmids were designed by us and constructed by VectorBuilder. These plasmids are available at Addgene.

### In Vitro Cell Cycle Assays

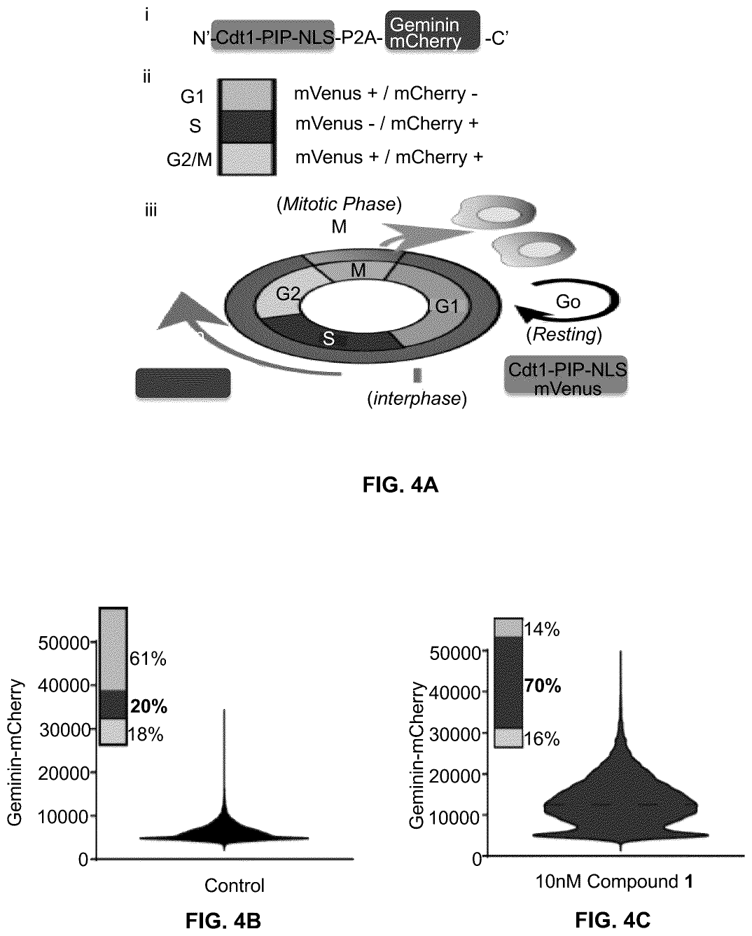

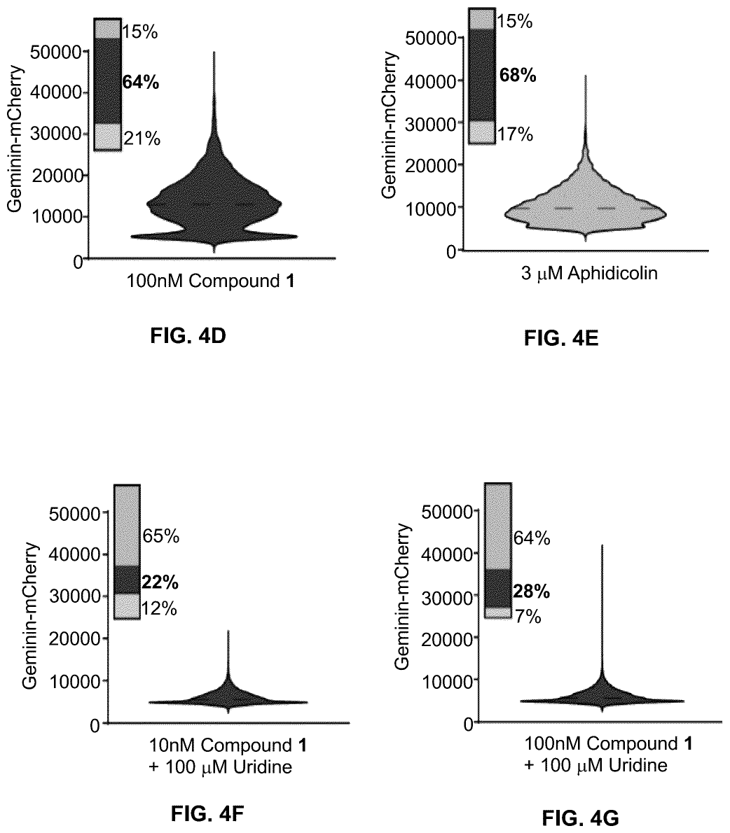

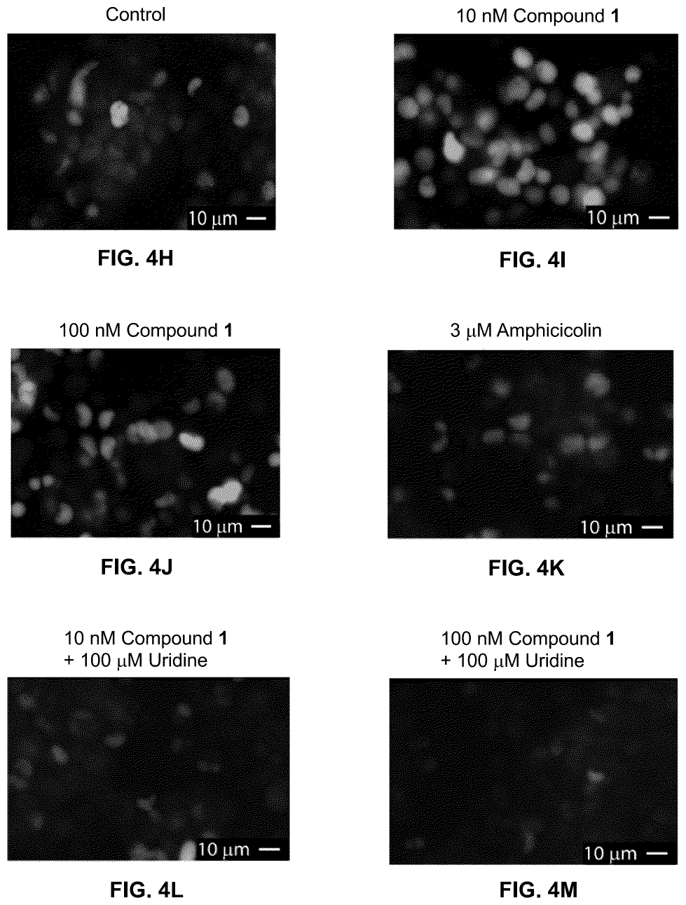

To monitor cell cycle progression, 913 glioblastoma neurosphere-derived cells expressing pLenti-PGK-Neo-PIP-FUCCI were first cloned. pLenti-PGK-Neo-PIP-FUCCI was from Jean Cook (Addgene plasmid #118616; n2t.net/addgene:118616; RRID:Addgene_118616) (52). Cells were then treated with either 0, 10 nM, or 100 nM compound 1 for 48 hours, stained with 1 mg/mL Hoechst for 2 hours, and visualized by direct fluorescence microscopy. A twenty-four-hour treatment with 3 mM of the DNA replication inhibitor Aphidicolin was used as a positive control for S-phase arrest. A collection of five images per well, each set containing at least 10,000 cells, was captured for each treatment. Each treatment was performed in technical quadruplicates, and each experiment was repeated twice on different days. Images were acquired using Cytation 5 plate reader (BioTek, Winooski, VT, USA) and scored using Gen5 software. Cell cycle phases were determined by x/y plot of mVenus and mCherry intensities per nucleus and gating boundaries on the Aphidicolin control. GraphPad 9 was used to calculate the described statistics and generate the violin plots presented in FIGS. 4B-4G.

### Immunofluorescence

The 913, 3691, 08-387, and 1079 glioblastoma neurosphere-derived cells (1×10⁴) were treated with DMSO (control) or IC50 concentration of compound 1 and cultured in either normoxia or hypoxia for 48 hours. Cells were spun with a Milles Scientific Cyto-Tek Model 4325 cytocentrifuge slide centrifuge onto Superfrost slides (VWR-48311-703) at 1500 rpm for 5 minutes. Slides were rinsed briefly with Hanks Balanced Salt Solution (HBSS), fixed in 4% paraformaldehyde in PBS, and rinsed with HBSS twice to remove any residual fixative. Cells were permeabilized with PBS containing 0.1% Triton-X100 (PBST) for 15 minutes, then blocked for 1 hour with PBST with 5% BSA. The slides were incubated overnight at 4° C. with Phospho-H2AX antibody (see above), diluted 1:250 in PBST containing 5% BSA. Slides were rinsed three times with HBSS, then incubated for one hour at room temperature with donkey anti-mouse, Alexa488-conjugated, secondary antibody diluted 1:500 in PBST containing 5% BSA (see above). Cell nuclei were counterstained with DAPI (150 nM), and the slides were cover-slipped with Prolong antifade mounting media (Molecular Probes #P36961). Using a 40X objective, at least ten random high-power fields, per condition, were captured with a Nikon W-1 Spinning Disk confocal microscope through the University of Maryland Baltimore CIBR confocal facility. The number of yH2AX-positive foci per cell was counted using ImageJ (unige.ch/medecine/bioimaging/files/3714/1208/5964/CellCounting.pdf). Individual cells with ten or more foci were considered positive for significant DNA damage. At least 300 cells were analyzed in each sample.

### Orthotopic Xenograft Transplantation and Drug Treatment

Experiments with animals were performed in compliance with institutional guidelines and regulations. Four- to six-week-old male and female NCG mice (NOD-Prkdcem26Cd52112rgem26Cd22/NjuCrl, Charles River Laboratory, Wilmington, MA) were used in all experiments in approximately equal numbers of each sex in each experimental group. The 08-387 m-Cherry Luciferase or 913 Luciferase expressing cells (1×10 5) were stereotactically injected into the right striatum as previously described (30). Two weeks after the injection of the cells, tumor growth was monitored with bioluminescence imaging using an IVIS Spectrum imaging system equipped with the Living Image software package (Perkin Elmer). Male and female mice harboring orthotopic xenografts with Radiance (p/sec/cm2/sr) equal to or greater than 1x10 6 were randomly assigned to the control or compound 1 treatment groups. Mice were dosed daily by oral gavage with either vehicle (90/10 Kollisolv® PEG400/EtOH solution) or 3 mg/kg compound 1. Animal weight was monitored daily, and mice were sacrificed when they became symptomatic.

### Statistical Methods

Unless otherwise noted, in vitro data are shown as mean values with error bars representing standard error of the mean for (at a minimum) three replicates, and experiments were repeated at least twice. Comparison of mean values between groups was evaluated by unpaired t-test and one or two-way ANOVA as appropriate. For all statistical methods, a p-value less than 0.05 was considered significant. Comparisons of survival curves were made using the log-rank test (Kaplan-Meier). All tests were performed using GraphPad Prism 9 (GraphPad Software, La Jolla, CA).

### Example 2

### Compound 1 Inhibits GBM Neurosphere Growth and Survival In Vitro by Inhibiting DHODH Activity

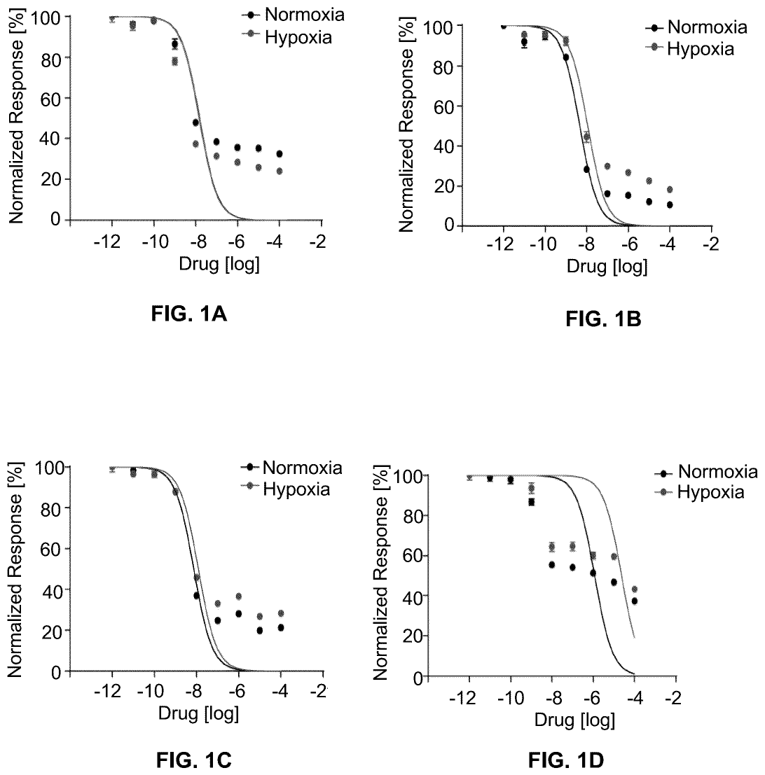

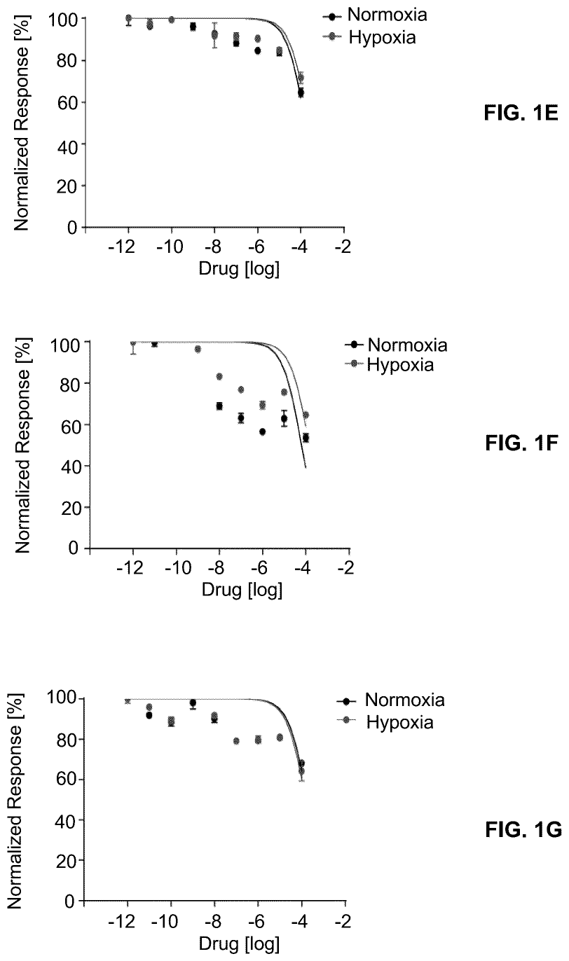

The compound's efficacy was examined against a collection of genetically diverse patient-derived GSC lines in vitro. The IC50 profiles of compound 1 in the seven GSC lines tested were established using the CellTiter-Glo® reagent and calculated with GraphPad software. Of the seven GSC lines, three were highly sensitive and included 913 (FIG. 1A), 08-387 (FIG. 1B), and 3691 (FIG. 1C) with IC50 values of 15.3 nM, 4.7 nM, and 7.4 nM, respectively. The 1079 GSC line exhibited moderate sensitivity (FIG. 1 D), with an IC50 value of 1.2 mM. On the other hand, Mayo6 (FIG. 1 E), Mayo15 (FIG. 1 F), and Mayo39 (FIG. 1G) were all refractory to drug treatment with calculated IC50 values of 164 mM, 64 mM, and 182 mM, respectively.

Because palisading necrosis and hypoxia-induced vascular proliferation are central to the pathological World Health Organization (WHO) classification of GBM tumors (22), these experiments in hypoxia were also performed. Hypoxic culture conditions significantly increased IC50 values in most of the neurosphere lines tested (growth inhibition curves in red).

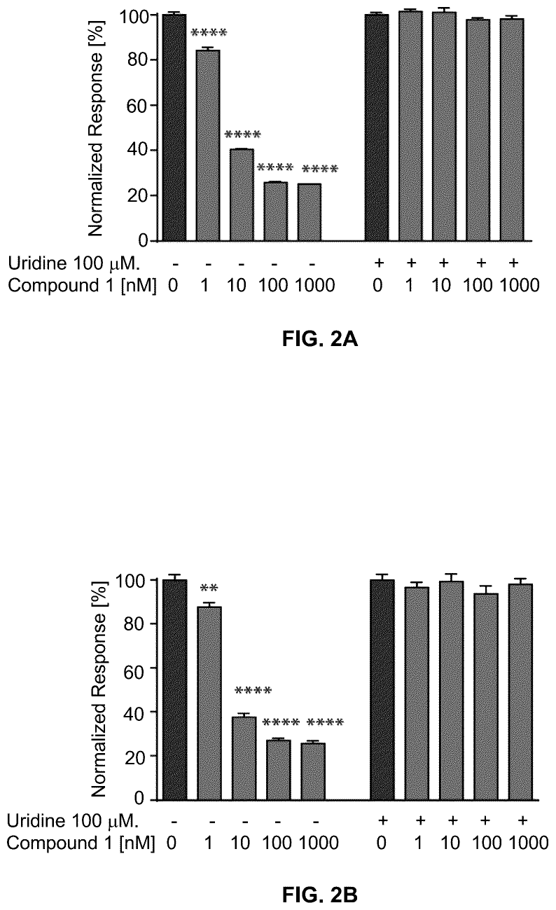

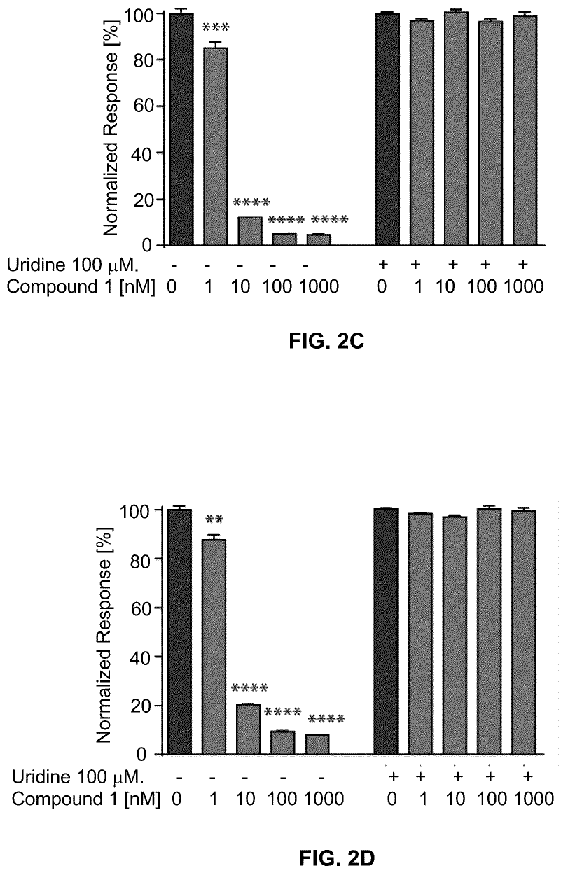

To confirm the specificity of compound 1 against de novo pyrimidine synthesis, the effects of dihydroorotate dehydrogenase inhibition after supplementing the growth medium with the nucleoside uridine using the drug-sensitive lines 913 and 08-387 (29) was shown. Cultured neurospheres were treated with increased compound 1 concentrations alone or in combination with 100 mM uridine. As in the previous experiments, these experiments were performed in normoxia (21% oxygen) and hypoxia (1% oxygen). Similar to the results shown in FIGS. 1A-1G, there was a dramatic and significant reduction in the number of viable cells in cultures treated with compound 1 alone with IC50 values between 1 and 10 nM for the 913 (FIGS. 2A-2B) and 08-387 (FIGS. 2C-2D) neurosphere lines. As in previous experiments, hypoxia slightly decreased the response to compound 1. Strikingly, 100 mM uridine entirely rescued the effects of compound 1 in both 913 and 08-387 against all the concentrations tested (FIGS. 2A-2D). Together, these experiments confirmed the on-target activity of compound 1 and suggested that reduced GSC survival in response to compound 1 is due to dihydroorotate dehydrogenase inhibition and reduced pyrimidine nucleotide availability.

### BAY2402234 Promotes DNA Damage

Reduced de novo pyrimidine synthesis mediated by MCT4-depletion promoted an accumulation of DNA damage (29). To test and quantify the magnitude of compound 1 effect on DNA damage, the four compound 1 responsive GBM neurosphere lines 913, 3691, 08-387, and 1079 were cultured in normoxia (21% oxygen) and hypoxia (1% oxygen) with a model-specific IC50 dose of compound 1 for 48 hours followed by immunocytochemical analysis of the DNA-damage marker g-H2AX. Table 1 is a summary of IC50 (nM or mM) values established for the seven glioblastoma neurosphere lines

TABLE 1
IC50 Dose of Compound 1 on GBM Neurosphere Lines

| | | |
|---|---|---|
| Cell Lines | Normoxia | Hypoxia |

| | | | | |
|---|---|---|---|---|
| 913 | 15.3 | nM | 6.7 | nM |
| 08-387 | 4.7 | nM | 11.4 | nM |
| 3639 | 7.4 | nM | 12.2 | nM |
| 1079 | 1.2 | μM | 23.7 | μM |
| Mayo6 | 164 | μM | 228 | μM |
| Mayo15 | 64 | μM | 146 | μM |
| Mayo39 | 182 | μM | 153 | μM |

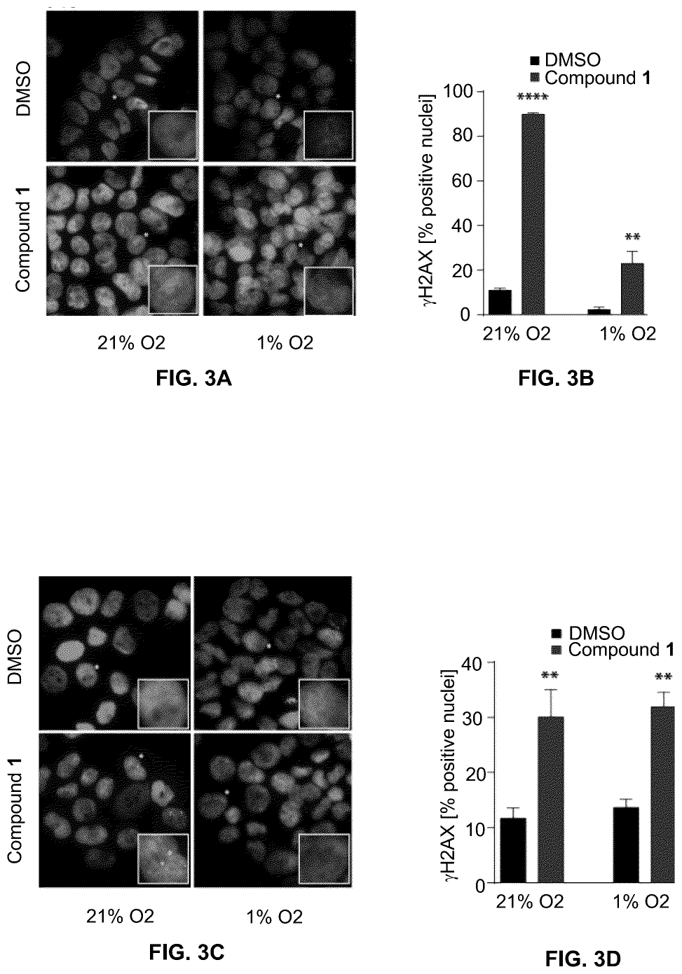

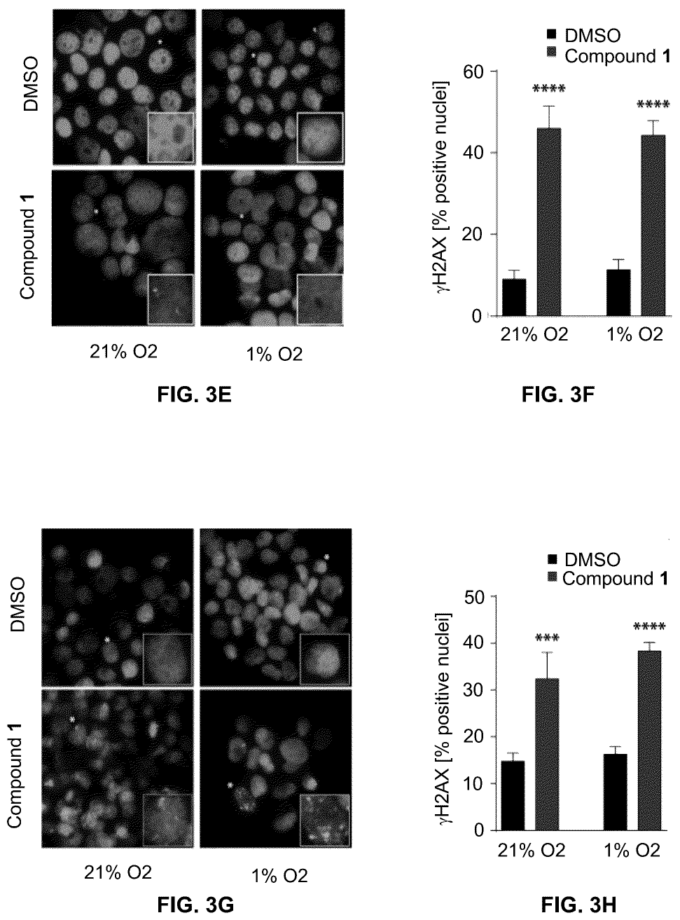

Treatment with compound 1 resulted in a dramatic and significant increase in the number of nuclei containing multiple yH2AX-positive foci (10, FIGS. 3A-3D), with 913 showing an average increase from 11% to 89% in normoxia and 2.3% to 23% in hypoxia (FIG. 3A, one-way ANOVA ****p<0.001, **p<0.01, respectively) and 08-387 showing an average increase from 11.6% to 30% in normoxia and 13.6% to 32% in hypoxia (FIG. 3B, one-way ANOVA, **p<0.01). In the 3691 neurosphere line, g-H2AX positivity increased from 9% to 46% in normoxia and from 11.3% to 44.2% in hypoxia (FIG. 3C, one-way ANOVA, ****p<0.0001). Finally, quantifying DNA damage in the 1079 neurosphere line showed a significant increase in g-H2AX positivity from 14.8% to 32.4% in normoxia and from 16.2% to 38.4% in hypoxia (FIG. 3D, one-way ANOVA, ****p<0.0001, ***p<0.001). These dramatic increases in DNA damage phenocopied the previously reported effects in MCT4-depleted GSCs (29) and further support the notion that the primary mechanism of growth inhibition in MCT4-depleted GSCs is pyrimidine nucleotide depletion.

Compound 1 Induces early S-Phase Cell Cycle Arrest

It has been reported that depletion of pyrimidine nucleotides leads to incomplete DNA replication and elicits a stress response that blocks cell cycle progression. To determine if compound 1 had a similar effect on GSC cycle progression, a modified version of the Fluorescent Ubiquitination-based Cell Cycle Indicator (FUCCI) reporter system was employed. The original design was developed in 2008 by Miyawaki and colleagues (51). It was more recently improved by Grant and colleagues, who developed a minimal FUCCI variant, PIP-FUCCI, that precisely indicates the transitions into and out of S-phase (FIG. 4A highlights the system's key features; also see (52). A 913 PIP-FUCCI reporter line was established and treated cells with DMSO (0.1% as control), 10 nM, or 100 nM compound 1.

As expected, the percentage of cells in the S phase increased from 20% to 70% and 64% in 10 nM and 100 nM compound 1 treated cells, respectively (FIGS. 4B-4D).

Notably, this over threefold increase was comparable with the effect of Aphidicolin, a cell-permeable tetracyclic diterpene antibiotic that blocks the cell cycle at the early S-phase (FIG. 4E). Consistent with compound 1 inhibition of dihydroorotate dehydrogenase activity, uridine supplementation completely blocked these cell cycle effects (FIG. 4F-4G). Finally, FIGS. 4H-4M shows representative images of these cell cycle analyses. Notably, the removal of compound 1 shortly after the synchronization of cells in the S-phase resulted in an almost complete reversal of the cell cycle arrest phenotype. These results suggest that persistent inhibition of dihydroorotate dehydrogenase is required to achieve a durable response.

### Compound 1 Inhibits GSC Growth in Methylcellulose-Supplemented Neurosphere Medium (NCMC)

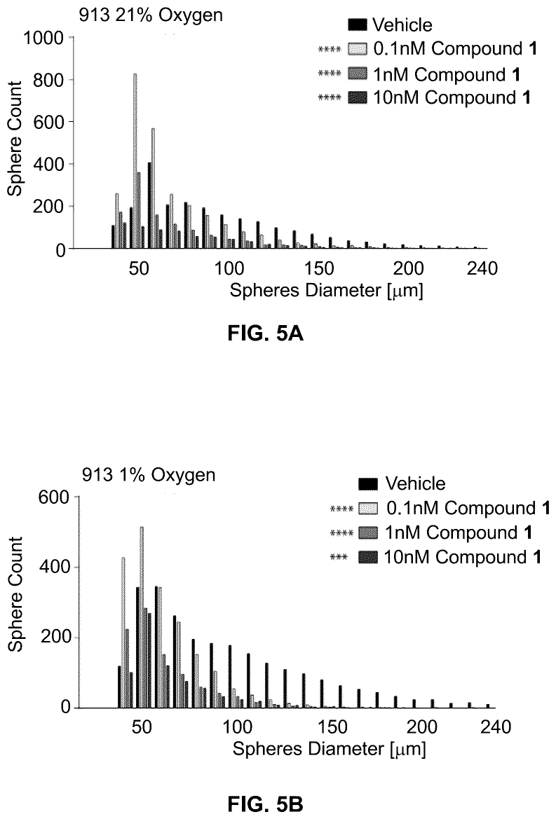

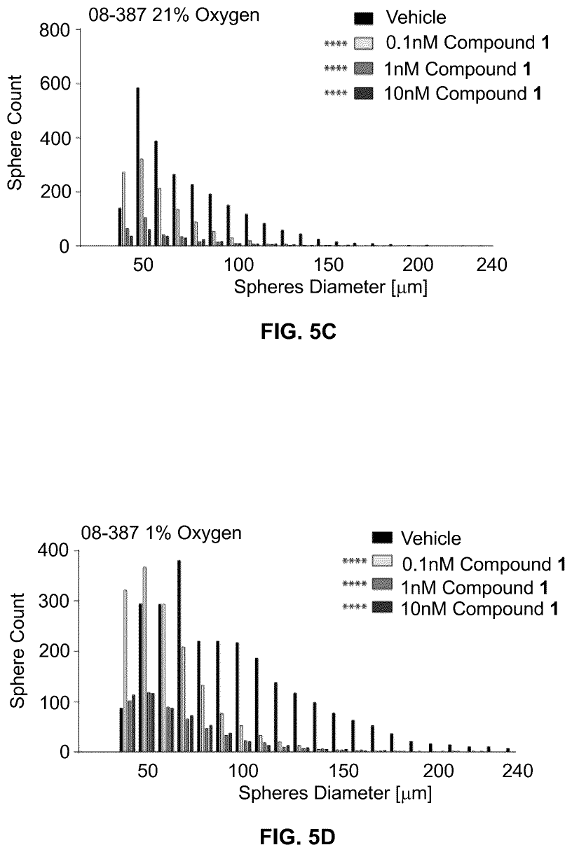

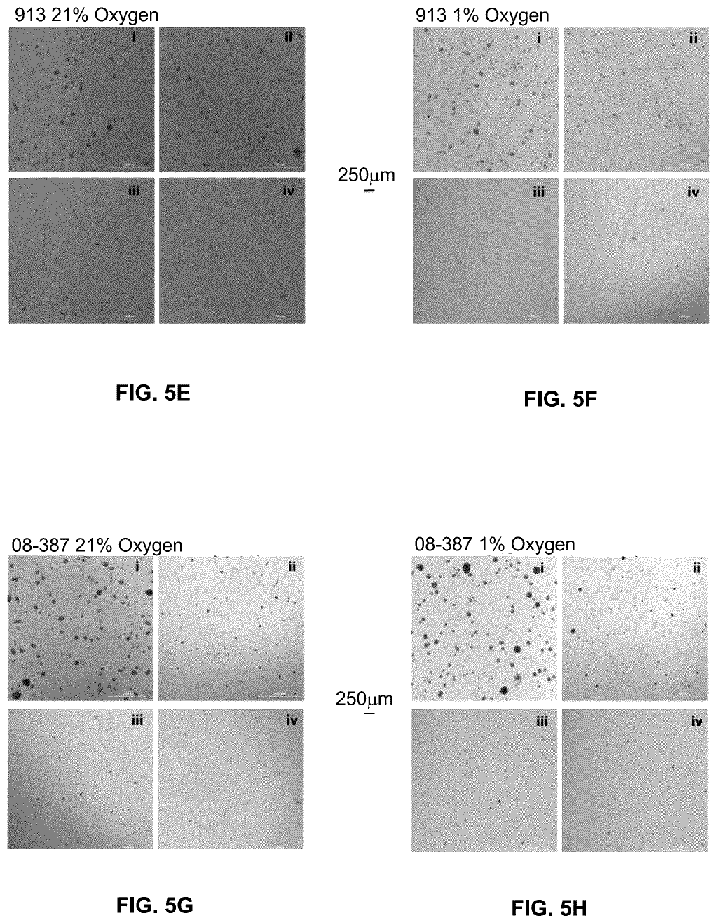

The effects of inhibition of de novo pyrimidine nucleotide synthesis on the growth of GBM neurospheres was measured in a methylcellulose-supplemented neural stem cell medium (NCMC). Neurospheres were dissociated to single-cell suspensions and incubated in normoxia (FIGS. 5A, 5C) or hypoxia (FIGS. 5B, 5D) with nM (0.1% DMSO), 0.1 nM, 1 nM, and 10 nM compound 1. After ten days of continuous treatment, the neurospheres were imaged, counted, and their diameter measured. compound 1 dramatically inhibited the growth of the 913 (FIGS. 5A, 5B, 5E, 5F) and 08-387 neurosphere lines (FIGS. 5C, 5D, 5G, 5H). In addition to a significant reduction in the overall sphere number, a significant reduction was also documented in sphere size (FIGS. 5A-D). In the 913 GBM neurosphere line, sphere diameter decreased from (DMSO) to an average diameter of 70 mm in compound 1 treated neurospheres cultured in normoxia (FIG. 5A) and from 76 mm to 63.3 mm when cultured in hypoxia (1% oxygen, FIG. 5B). Similarly, in the 08-387 GBM neurosphere line, sphere diameter decreased from 97.6 mm (DMSO) to an average diameter of 61.5 mm in compound 1 treated neurospheres cultured in normoxia (FIG. 5C) and from 95 mm to 66.5 mm when cultured in hypoxia (1% oxygen, FIG. 5D). Finally, representative low-power images of treated neurospheres are shown in FIGS. 5E-5H.

### Compound 1 Induces Programmed Cell Death

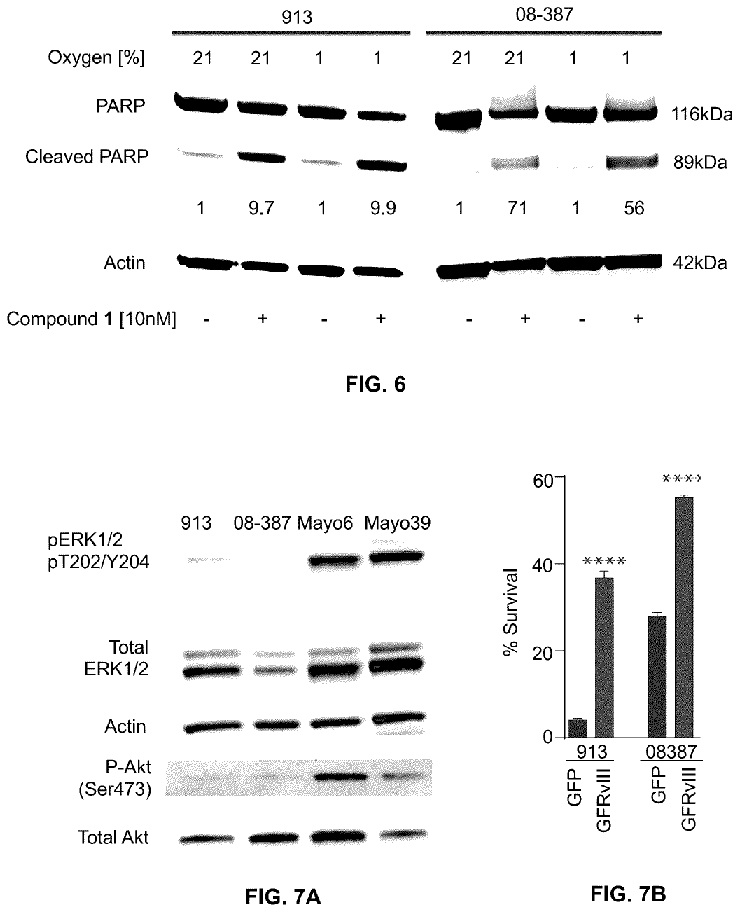

Poly(ADP-ribose) polymerase 1 (PARP1) is a nuclear protein that is activated by binding to DNA lesions and catalyzes the poly(ADP-ribosyl)ation of various nuclear proteins, including PARP1 itself, to recruit the DNA repair machinery to DNA lesions (53, 54. When excessive DNA damage occurs, PARP1 is cleaved to generate an 89 kDa fragment shown to serve as a cytoplasmic PAR carrier to induce AIF-mediated apoptosis (55). Given the profound induction of DNA damage in compound 1-treated GBM neurospheres, the prevalence of the 89 kDa PARP1 fragment in control and 10 nM compound 1-treated cells was measured. Strikingly, a 9.7- and 56-fold increase in the level of cleaved PARP1 was found in 913 and 08-387 neurosphere lines, respectively, when cultured in normoxia. Similarly, a 9.9- and 71-fold increase was documented in the level of cleaved PARP1 in 913 and 08-387 neurosphere lines cultured in hypoxia (FIG. 6).

### Epidermal Growth Factor Receptor Amplification Confers Resistance to De Novo Pyrimidine Synthesis Inhibition

Examining the Mayo Clinic database for genetic alterations that may explain the strong resistance of the PDX models in this study to compound 1, EGFR or its activated variant, EGFRvIll, are amplified in all three models (Mayo 6, 15, and 39). To determine if EGFR may be involved in compound 1 resistance, ERK's and AKT's steady state phosphorylation state as EGFR signaling surrogates was first examined.

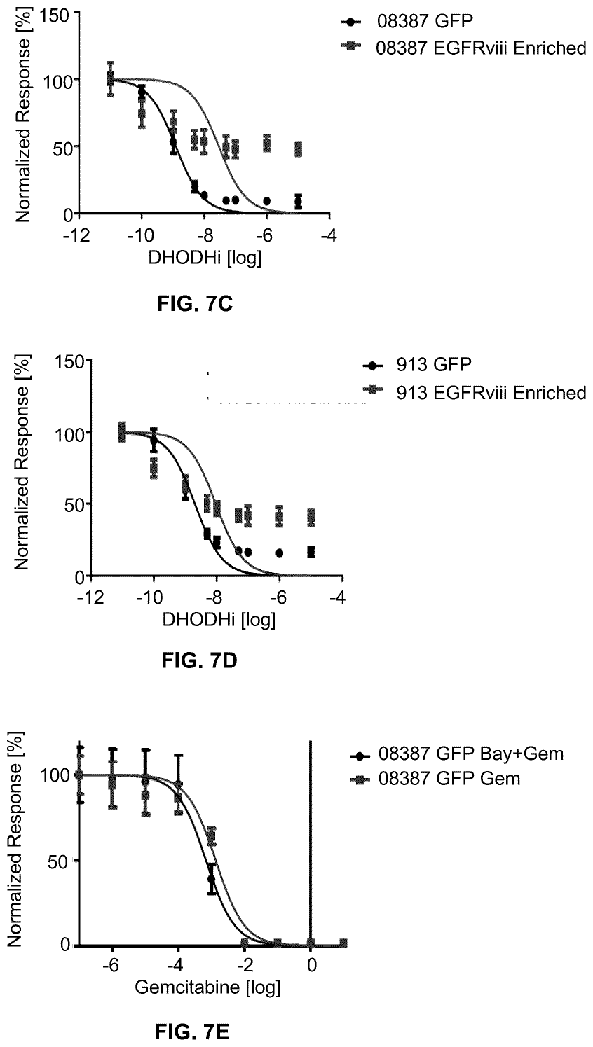

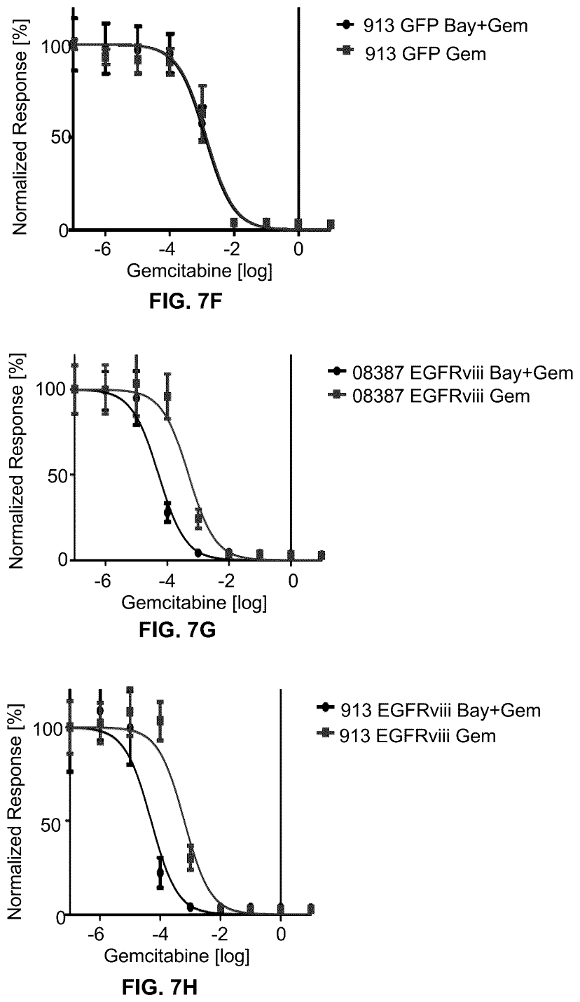

To this end, Western blot analyses for total and phospho- ERK and AKT were performed on lysates from 913, 08-387, Mayo 6, and Mayo 39 neurospheres. FIG. 7A shows that MAPK and PI3K pathways are constitutively active in compound 1 resistant but not in responsive lines.

To further evaluate the potential role of EGFR in compound 1 resistance, the 913 and 08-387 neurosphere lines were transduced with lentiviruses encoding EGFRvIll or GFP (as control) and these neurospheres were challenged with a relatively high dose of 1 mM compound 1. EGFRvIll increased the surviving cell fraction from 27.9% to 55.2% and from 4% to 36.7% in 913 and 08-387 neurosphere lines, respectively (FIGS. 7B-7D). This set of experiments demonstrates that EGFR signaling is constitutively active in compound 1 resistant GSCs and that EGFRvIll is sufficient to impart compound 1 resistance in otherwise responsive models. Moreover, assaying the EGFRviii expressing 08-387 and 913 glioblastoma cells treated with varying dosages of gemcitabine and 0.05 nM of compound 1 (FIGS. 7E-7F) or with varying dosages of gemcitabine and 100 nM of compound 1 (FIGS. 7G-7H) demonstrates, respectively, that these glioblastomas are sensitive to gemcitabine and that compound 1 sensitizes EGFRviii expressing glioblastomas to gemcitabine.

### Example 3

### Compound 1 Administration Inhibits Intracranial Tumor Progression and Prolongs Mouse Survival

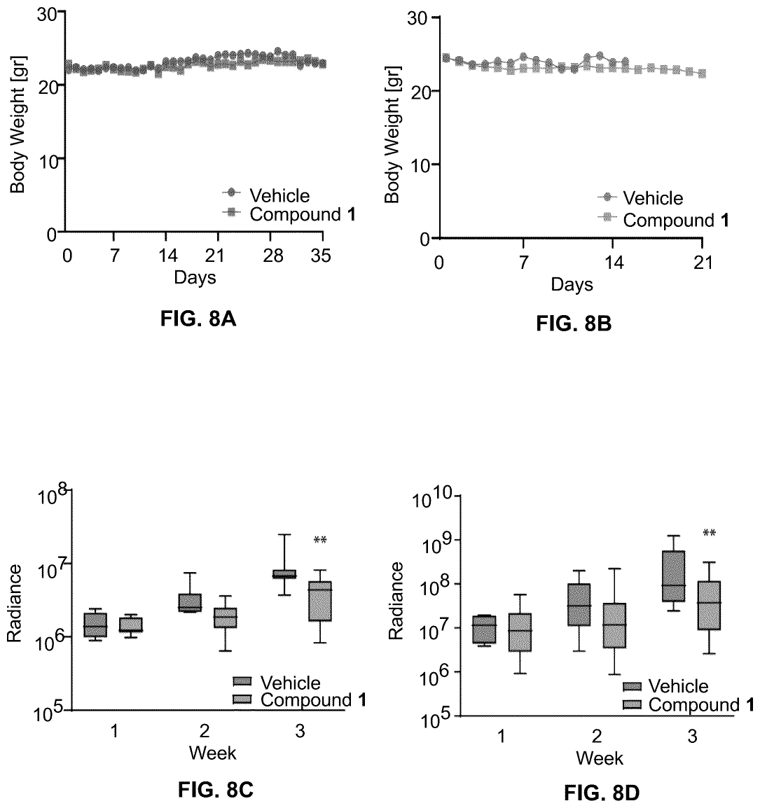

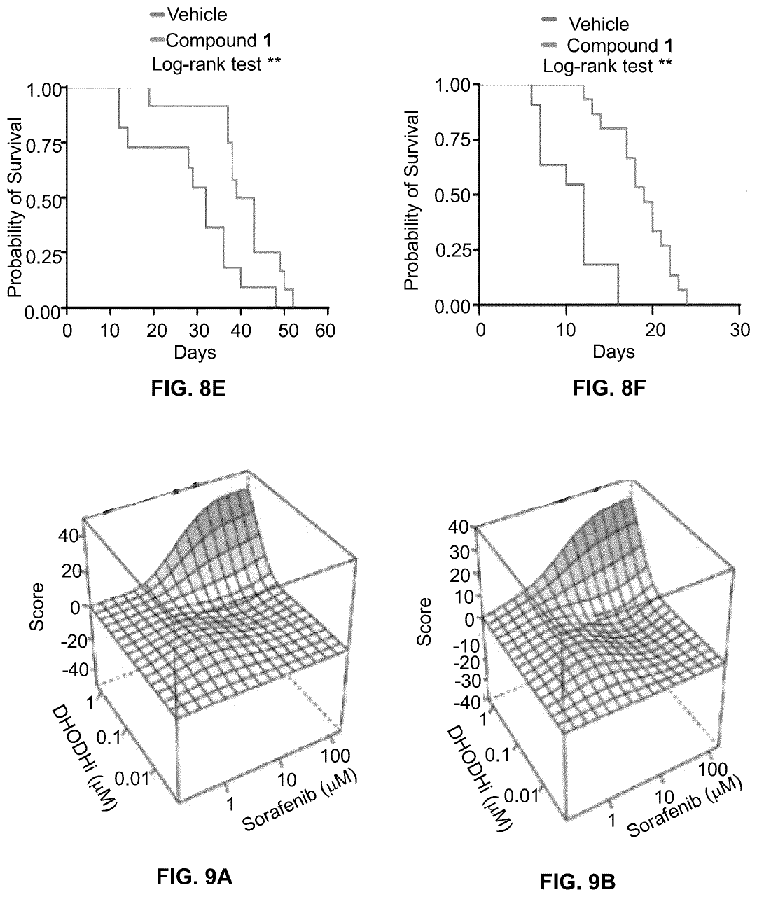

Orthotopic (intracranial) tumor xenografts were initiated using 913 and 08-387 GBM-derived neurosphere lines which have been shown to form infiltrative and highly proliferative malignant gliomas in immune-deficient mice. To generate equivalent treatment groups and facilitate the tracking of xenograft growth, these cells were transduced with a mCherry:Luciferase reporter. An equal number of male and female immunodeficient mice were injected, and overall survival was the primary endpoint. Mice were imaged weekly starting two weeks after the injection and then randomized to the two treatment groups when tumor Radiance surpassed 1x10 6 photons/sec/cm²/sr (sr=units of solid angle or steradian). Compound 1 (3 mg/kg) or vehicle were delivered daily by oral gavage. Mouse body weight was monitored daily, just before treatment, and no significant weight loss was noted in the compound 1-treated animals as compared with controls until they became symptomatic due to increased tumor burden, suggesting that daily administration of this dihydroorotate dehydrogenase inhibitor was well-tolerated (FIGS. 8A-8B).

Weekly bioluminescence imaging revealed growth in the control and compound 1 treatment groups (FIGS. 8C-8D). However, xenografts in the BAY2402234-treated group were consistently smaller, as indicated by lower tumor radiance. In 913, tumor radiance in the first three weeks of treatment was 6.5%, 41%, and 52% lower in compound 1-treated animals than in vehicle controls (**p<0.01, mixed-effects model analysis with . 8 idak's multiple comparisons test, FIG. 8C). Similarly, 08-387 tumor radiance was roughly the same on week 1, then decreased to 47% and 75% lower on weeks 2 and 3, respectively, in compound 1, treated animals as compared to controls (**p<0.01, mixed-effects model analysis with . 8 idak's multiple comparisons test, FIG. 8D). These data suggest that compound 1 effectively slowed tumor progression throughout the experiments in the 913 and 08-387 orthotopic xenograft models.

Finally, median survival from the point treatment was initiated was also significantly longer in animals receiving daily compound 1. In mice bearing 913 xenografts, median survival increased 28% from 32 days to 41 days (log-rank test ** p <0.01, FIG. 8E). Median survival in mice bearing 08-387 xenografts increased 58% from 12 to 19 days (log-rank test ****p<0.0001, FIG. 8F). Thus, dihydroorotate dehydrogenase inhibition by compound 1 therapy can slow the orthotopic growth of these two genetically distinct patient-derived GBM neurosphere lines and prolong animal survival.

### Example 4

EGFR or Mutant EGFRvIll Overexpression in Nestin-Expressing Cells in Combination with Other Glioma Driving Alterations Promote DHODHi Resistance In Vivo

Adult Nestin-TVA (Ntv-a) mice bearing de novo GBMs are used to determine if adding EGFR wildtype or EGFRvIll to a combination of alterations that promote GBM will result in the generation of DHODHi-resistant tumors. To this end, twenty mice (male and female mice in equal proportions from each of three genetic backgrounds) are included in each treatment group (DHODHi or vehicle) for a total of 120 mice (3 oncogenic drivers x 2 treatment groups x 20 mice per group=120). Animals are dosed daily by oral gavage with either vehicle or 4 mg/kg DHODHi (80% of MTD). Treatment will begin 2-3 weeks after RCAS transduction (guided by bioluminescence imaging).

Two hours after the second drug administration (DHODHi half-life is 4.1 hr), four animals from each control and DHODHi treatment group are sacrificed, and their brains removed. Pyrimidine levels and orotate levels are measured in tumor tissues as a surrogate marker for DHODH activity. A second portion of each tumor is used to initiate neurospheres to be challenged in vitro with DHODHi. The third portion is frozen for molecular analyses that include: (1) Western blot analyses to gauge the activation state of relevant pathways; (2) qPCR validation of nucleoside transporters and multidrug resistance proteins. (3) Immunofluorescence staining to validate critical markers such as Nestin, EGFR, SOX2 and GFAP. (4) Explore additional markers of response and resistance by RNA sequencing coupled with gene set enrichment analysis.

The remaining animals undergo weekly bioluminescence imaging to monitor tumor burden and survival analysis as the primary endpoint. All animals are sacrificed when they become symptomatic and two hours from the last treatment and undergo the same procedures described above to determine if treatment failure/tumor progression results from lack of drug penetration or drug resistance. Here, elevated orotate levels would suggest a lack of drug penetration (the enzyme is no longer inhibited), while a sustained reduction in orotate levels may indicate resistance (the enzyme is still inhibited, but the tumor continues to grow).

### Combination Treatment Strategies to Overcome DHODHi Resistance In Vitro

Support for the involvement of ERK signaling in DHODHi resistance is presented in FIG. 6, where the DHODHi sensitive GSC lines 913 and 08-387 exhibit low ERK1/2 phosphorylation levels as compared with Mayo 6 and Mayo 39. To demonstrate that Sorafenib sensitizes GBMs to DHODHi, Mayo 6 and Mayo 39 neurospheres were treated with doses of the two drugs alone and in combination. These experiments established a synergistic relationship between the two drugs, with the most synergistic area score of 8.39 and 10.48 for Mayo 39 and Mayo 6, respectively (FIGS. 9A-9B).

To identify potential small molecules that can synergize with DHODHi, the Approved Oncology Drugs Set (NIH Developmental Therapeutics Program) was screened alone and combined with DHODHi (10 μM) against Mayo 6. Several drugs were identified that dramatically inhibited growth when combined with DHODHi (Table 2). These included: vandetanib (Caprelsa, VEGFR, EGFR, and RET), vemurafenib (Zelboraf, BRAF), regorafenib (Stivarga, dual inhibitor VEGFR-TIE2), and osimertinib (Tagrisso, EGFR). These results showing synergistic interaction between DHODHi and sorafenib and the identification of drugs targeting EGFR, VEGFR, and BRAF suggest that inhibition of EGFR, signaling downstream of EGFR, and other receptor tyrosine kinases, sensitize group a GBMs to DHODHi.

TABLE 2
Synergism of DHODH Inhibitors (DHODHi)
with other small molecule drugs

| Drug Name | Target(s) | DHODHi | Alone | Combination |
|---|---|---|---|---|
| Vandetanib | VEGFR, EGFR, | 1.00 | 0.79 | 0.04 |
| | and RET | | | |
| Vermurafenib | BRAF | | 0.93 | 0.72 |
| Regorafenib | VEGFR-TIE2 | | 1.00 | 0.64 |
| Osimertinib | EGFR | | 0.35 | 0.0001 |

Compounds and additional inhibitors such as Erlotinib (EGFR), Gefitinib (EGFR), Trametinib (GSK1120212, reversible allosteric inhibitor of MEK1 and MEK2 activity), MK-8353 (potent and selective ERK1/2 inhibitor), Ravoxertinib (GDC-0994, inhibits both ERK phosphorylation and activation of ERK-mediated signal transduction pathways), and Ipatasertib (GDC-0068, a highly selective and potent pan-Akt inhibitor) are synergistic with DHODHi.

Combination of DHODHi with DHODHi Sensitizing Agents Against De Novo Murine GBMs Overexpressing EGFR or Mutant EGFRviii

Adult Nestin-TVA mice bearing de novo GBMs are used to determine the synergization of DHODHi sensitizing agents with DHODHi. Ntv-a: EGFR fl/fl; Ink4a-Arf-/-; Pten fl/fl mice and Ntv-a: EGFRvIll fl/fl; Ink4a-Arf-/-; Pten fl/fl transgenic mice are used. Twenty adult mice (male and female mice in equal proportions) are included in each of four treatment groups (Vehicle, DHODHi, DHODHi sensitizing agent, and the combination of DHODHi and DHODHi sensitizing agent) for a total of 240 mice (3 genetic backgrounds x 2 treatment groups x 20 mice per group=240). These studies demonstrate that DHODHi sensitizing agents synergize with DHODHi to inhibit the growth of de novo murine GBMs overexpressing EGFR or mutant EGFRvIll.

The following references are cited herein:

1. Hess et al. Cancer 101:2293-2299 (2004).

2. Stupp et al. N Engl J Med 352:987-996 (2005).

3. Woodward et al. Proc Natl Acad Sci USA, 104:618-623 (2007).

4. Wang et al. Int J Oncol, 43:131-140 (2013).

5. Wang et al. Stem Cells, 28:17-28 (2010).

6. Uribe et al. Mol Aspects Med 55:140-151 (2017).

7. J. N. Rich, Cancer Res, 67:8980-8984 (2007).

8. S. Osuka and E. G. Van Meir, J Clin Invest, 127:415-426 (2017).

9. R. M. A. I. Nakano, J Carcinogene Mutagene, 8 (2011).

10. Kim et al. Lab Invest 92, 466-473 (2012).

11. Wang et al. Stem Cells, 28:17-28 (2010).

12. Honoki et al. Oncol Rep, 24:501-505 (2010).

13. Hardee et al. Cancer Res 72, 4119-4129 (2012).

14. Facchino et al. J Neurosci 30, 10096-10111 (2010).

15. Eyler et al. J Clin Oncol 26, 2839-2845 (2008).

16. Diehn et al. Nature 458, 780-783 (2009).

17. Dean et al. Nat Rev Cancer 5, 275-284 (2005).

18. Dahan et al. Cell Death Dis 5, e1543 (2014).

19. Carruthers et al. Cancer Res 78, 5060-5071 (2018).

20. Bao et al. Nature 444, 756-760 (2006).

21. Szeto et al. Cancer Res 69, 4502-4509 (2009).

22. G.N. Fuller, Arch Pathol Lab Med 132:906 (2008).

23. Heddleston et al. Cell Cycle 8:3274-3284 (2009).

24. Bar et al. Am J Pathol, 177:1491-1502 (2010).

25. Pistollato et al. Stem Cells, 27:7-17 (2009).

26. Li et al. Cancer Cell, 15:501-513 (2009).

27. Seidel et al. Brain 133:983-995 (2010).

28. Soeda et al. Oncogene 28:3949-3959 (2009).

29. Spina et al. Neurooncol Adv, 2:vdz062 (2020).

30. Bar et al. Stem Cells, 25:2524-2533 (2007).

What is claimed is:

1. A method for treating a brain cancer in a subject in need thereof, comprising:
administering at least once to the subject a pharmacologically effective amount of an inhibitor of dihydroorotate dehydrogenase.

2. The method of claim 1, further comprising administering to the subject a pharmacologically effective amount of at least one small molecule drug or pharmaceutically acceptable compositions thereof synergistic with the inhibitor.

3. The method of claim 2, wherein the small molecule drug is gemcitabine, sorafenib, vandetanib, vemurafenib, regorafenib, or osimertinib.

4. The method of claim 2, wherein the at least one small molecule drug is co-administered with the inhibitor or is administered sequentially therewith.

5. The method of claim 1, further comprising administering at least once to the subject a chemoradiation therapy.

6. The method of claim 1, wherein the inhibitor of dihydroorotate dehydrogenase is compound 1 with a chemical structure of: 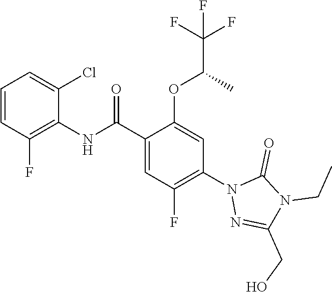 [SMILES: CCn1c(CO)nn(-c2cc(O[C@@H](C)C(F)(F)F)c(C(=O)Nc3c(F)cccc3Cl)cc2F)c1=O]
or a pharmaceutically acceptable composition thereof.

7. The method of claim 1, wherein the brain cancer is a malignant glioma.

8. The method of claim 7, wherein the malignant glioma is a glioblastoma.

9. The method of claim 8, wherein the glioblastoma overexpresses EGFR or mutant EGFRviii.

10. A method for decreasing proliferation of malignant glioma tumor cells, comprising:
contacting the glioma tumor cells with at least one small molecule drug that inhibits an activity of dihydroorotate dehydrogenase or an activity associated therewith.

11. The method of claim 10, wherein the contacting step is in vivo, the method comprising administering at least once to a subject in need thereof a pharmacologically effective amount of the small molecule drug.

12. The method of claim 10, wherein the small molecule drug is compound 1 with a chemical structure of: 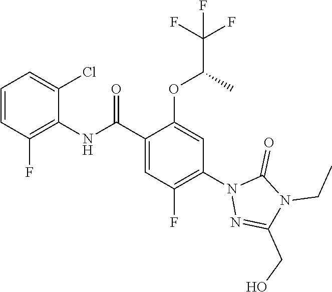 [SMILES: CCn1c(CO)nn(-c2cc(O[C@@H](C)C(F)(F)F)c(C(=O)Nc3c(F)cccc3Cl)cc2F)c1=O]
or a pharmaceutically acceptable composition thereof.

13. The method of claim 12, wherein the small molecule drug comprises a combination of compound 1 and at least one other small molecule drug synergistic therewith or a pharmaceutically acceptable composition of the other small molecule drug.

14. The method of claim 13, wherein the at least one other small molecule drug is gemcitabine, sorafenib, vandetanib, vemurafenib, regorafenib, or osimertinib.

15. The method of claim 10, further comprising administering at least once to the subject a chemoradiation therapy.

16. The method of claim 10, wherein the contacting step is in vitro, the method comprising bringing the malignant glioma tumor cells into contact with compound 1 or a combination of compound 1 and at least one of gemcitabine, sorafenib, vandetanib, vemurafenib, regorafenib, or osimertinib.

17. The method of claim 10, wherein the malignant glioma tumor cells comprise a glioblastoma.

18. The method of claim 17, wherein the glioblastoma overexpresses EGFR or mutant EGFRviii.

19. A method for increasing sensitivity of a malignant glioma to a drug regimen for a subject in need thereof, comprising:
administering to the subject at least once a regimen of compound 1 with a chemical structure of: 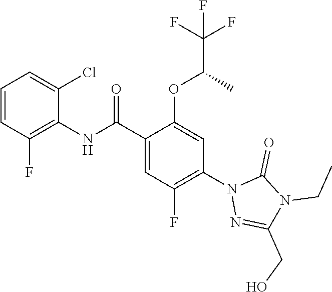 [SMILES: CCn1c(CO)nn(-c2cc(O[C@@H](C)C(F)(F)F)c(C(=O)Nc3c(F)cccc3Cl)cc2F)c1=O]
and at least one of a small molecule drug that synergizes therewith or pharmaceutically acceptable compositions thereof.

20. The method of claim 19, wherein the at least one small molecule drug is gemcitabine, sorafenib, vandetanib, vemurafenib, regorafenib, or osimertinib.

21. The method of claim 19, wherein the drug regimen comprises co-administering or sequentially administering compound 1 and the at least one small molecule drug or the pharmaceutically acceptable compositions thereof.

22. The method of claim 19, wherein the malignant glioma is a glioblastoma.

23. The method of claim 20, wherein the glioblastoma overexpresses EGFR or mutant EGFRviii.
